(19) United States
(12) Patent Application Publication
(10) Pub. No.: US 2014/0073893 A1
(43) Pub. Date: Mar. 13, 2014

(54) OPEN IRRIGATED-MAPPING LINEAR ABLATION CATHETER

(71) Applicant: Boston Scientific Scimed Inc., Maple Grove, MN (US)

(72) Inventor: Robert F. Bencini, Sunnyvale, CA (US)

(21) Appl. No.: 13/916,786

(22) Filed: Jun. 13, 2013

Related U.S. Application Data
(60) Provisional application No. 61/700,235, filed on Sep. 12, 2012.

(51) Int. Cl. A61B 18/14; A61B 18/18; A61B 5/042; A61B 5/00; A61M 5/00; A61M 25/00

(52) U.S. Cl. CPC A61B 18/1442; A61B 18/1492; A61M 5/00; A61M 25/0082; A61B 5/042; A61B 5/4836; A61B 18/18
USPC 600/374; 606/46; 606/33

(57) ABSTRACT

A catheter device for use in ablating heart tissues includes an elongate body having a proximal end and an opposite distal end, and a tip section positioned at the distal end of the elongate body. The tip section includes a first jaw member and a second jaw member each including a proximal portion, a distal portion, an outer surface, and an inner surface. The jaw members are pivotally joined to one another at the proximal portions thereof, and the tip section is configured to transition between a closed configuration in which the inner surfaces are at least partially in contact with one another, and an open configuration in which the distal portions of the jaw members are deflected away from one another. The tip section is operable as ablation electrode for selectively ablating the heart tissues.

### CROSS-REFERENCE TO RELATED APPLICATION

This application claims priority to Provisional Application No. 61/700,235, filed Sep. 12, 2012, which is herein incorporated by reference in its entirety.

### TECHNICAL FIELD

The present disclosure generally relates to systems and methods for providing a therapy to a patient. More particularly, the present disclosure relates to a catheter for mapping and ablating tissue within the heart of the patient.

### BACKGROUND

Various catheters have been developed for use in ablating cardiac tissue proximate the pulmonary vein ostia in the left atrium in an effort to treat atrial fibrillation. Such catheters include capabilities for mapping bioelectrical signals arising proximate these ostia. There remains a continuing need for improvements in the foregoing catheters.

### SUMMARY

In Example 1, a catheter device for use in ablating heart tissues. The catheter device comprises an elongate body and a tip section. The elongate body has a proximal end and an opposite distal end. The tip section is positioned at the distal end of the elongate body, and includes a first jaw member and a second jaw member. Each of the jaw members includes a proximal portion, a distal portion, an outer surface, and an inner surface, the jaw members pivotally joined to one another at the proximal portions thereof. The tip section is configured to transition between a closed configuration in which the inner surfaces are at least partially in contact with one another, and an open configuration in which the distal portions of the jaw members are deflected away from one another. In addition, the tip section is operable in both the open and closed configurations as an ablation electrode for selectively ablating the heart tissues.

In Example 2, the catheter device of Example 1, wherein the proximal portions of the first jaw member and the second jaw member are coupled together by a hinge element to allow splitting of the tip section by allowing the distal portions of the first jaw member and the second jaw member to deflect away from one another.

In Example 3, the catheter device of Examples 1 or 2, wherein the catheter is an irrigated catheter configured to deliver irrigation fluid through the tip section during an ablation procedure.

In Example 4, the catheter device of any of Examples 1-3, wherein the tip section is configured to map electrical activity of the heart.

In Example 5, the catheter device of any of Examples 1-4, wherein the first jaw member and the second jaw member are substantially perpendicular with respect to the elongate body in the open configuration.

In Example 6, the catheter device of any of Examples 1-5, wherein the first jaw member and the second jaw member configured to lie linearly in the open configuration.

In Example 7, the catheter device of any of Examples 1-6, wherein the inner surfaces of the first and second jaw members are generally flat.

In Examples 8, the catheter device of any of Examples 1-7, further comprising a control mechanism coupled to the tip section for manipulating the tip section between its closed and open configurations.

In Example 9, the catheter device of Example 8, wherein the control mechanism includes first and second control members connected, respectively, to the first and second jaw members and slideably disposed within the elongate body.

In Example 10, the catheter device of Examples 8 or 9, further comprising a handle coupled to the proximal end of the body, wherein the control mechanism includes a control element on the handle, and wherein the first and second control members extend at least partially within the elongate body from the tip section to the handle and are coupled to the control element, and wherein manipulation of the control element causes the control members to move relative to the elongate body so as to cause the tip section to transition between its open and closed configuration.

In Example 11, the catheter device of any of Examples 8-10, wherein the control mechanism is configured such that proximal movement of the first and second control members relative to the elongate body causes the distal portions of the first and second jaw members to deflect away from one another thereby causing the tip section to assume its open configuration.

In Example 12, the catheter device of any of Example 8-11, wherein the control mechanism includes a locking element to releasably retain the tip section in its open configuration at the target location within the patient.

In Example 13, a catheter device for use in ablating heart tissues. The catheter device comprises an elongate body and a tip section. The elongate body has a proximal end and an opposite distal end, and an inner fluid lumen extending from the proximal end through the distal end of the body. The tip section includes a first jaw member, a second jaw member, a plurality of microelectrodes, and a plurality of irrigation ports. The tip section is positioned at the distal end of the elongate body. Each of the jaw members includes a proximal portion, a distal portion, an outer surface, and an inner surface, the jaw members pivotally joined to one another at the proximal portions thereof. The tip section is configured to transition between a closed configuration in which the inner surfaces are at least partially in contact with one another, and an open configuration in which the distal portions of the jaw members are deflected away from one another, wherein the tip section is operable as an ablation electrode for selectively ablating the heart tissues. The microelectrodes are positioned on the inner surface of each of the jaw members for sensing electrical signals originating from the heart tissues. The irrigation ports provided on the inner surface of each of the jaw members and in fluid communication with the fluid lumen.

In Example 14, the catheter device of Example 13, wherein, the first jaw member and the second jaw member are substantially perpendicular with respect to the elongate body in the open configuration.

In Example 15, the catheter device of Example 13 or 14, wherein the first jaw member and the second jaw member configured to lie linearly in the open configuration.

In Example 16, the catheter device of any of Examples 13-15, wherein the inner surfaces of the first and second jaw members are generally flat.

In Example 17, the catheter device of any of Examples 13-16, further comprising a control mechanism coupled to the tip section for manipulating the tip section between its closed and open configurations, and further wherein the tip section is biased to its closed configuration, and wherein manipulation of the control mechanism causes the tip section to assume its open configuration.

In Example 18, a tissue ablation method comprising first advancing a portion of a catheter device to a location proximate target tissue within a patient's heart. The catheter device includes a tip section positioned at a distal end of an elongate body of the catheter device, the tip section including a first jaw member and a second jaw member, each of the jaw members including a proximal portion, a distal portion, an outer surface, and an inner surface. The jaw members are pivotally joined to one another at the proximal portions thereof. The advancing step is performed with the tip section in a closed configuration in which the inner surfaces are at least partially in contact with one another. The method further comprises manipulating a control mechanism on the catheter device to cause the distal portions of the jaw members to deflect and rotate away from one another such that the tip section assumes an open configuration wherein the inner surfaces of the jaw members are exposed to and positioned proximate the target tissue. The method further comprises positioning the inner surfaces of the jaw members in contact with the target tissue, and causing RF ablation energy to be supplied to the jaw members to effectuate RF ablation of the target tissue in contact with the inner surfaces of the jaw members.

In Example 19, the method of Example 18, further comprising, after manipulating the control mechanism to cause the tip section to assume its open configuration, mapping cardiac electrical activity proximate the target tissue using microelectrodes positioned on the inner surfaces of the jaw members.

In Example 20, the method of Example 18 or 19, further comprising causing irrigation fluid to be supplied to the target tissue while supplying the RF ablation energy via irrigation ports disposed in the inner surfaces of the jaw members.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram illustrating an example of a catheter device positioned in a pulmonary vein of a heart.

FIG. 2 is a schematic elevation view of the catheter device of FIG. 1, in accordance with an embodiment.

FIGS. 3A-3B are schematic elevation views of a portion of the catheter device of FIG. 2, showing a tip section of the catheter in closed and open configurations, respectively, according to an embodiment.

FIG. 4 is an end view of the catheter of FIG. 2 with the tip section in the open configuration shown in FIG. 3B.

FIG. 5 is a schematic elevation view of a portion of an alternative embodiment of the catheter device of FIG. 2, showing the tip section of the catheter in the open configurations.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as falling within the scope of the claims, together with all equivalents thereof.

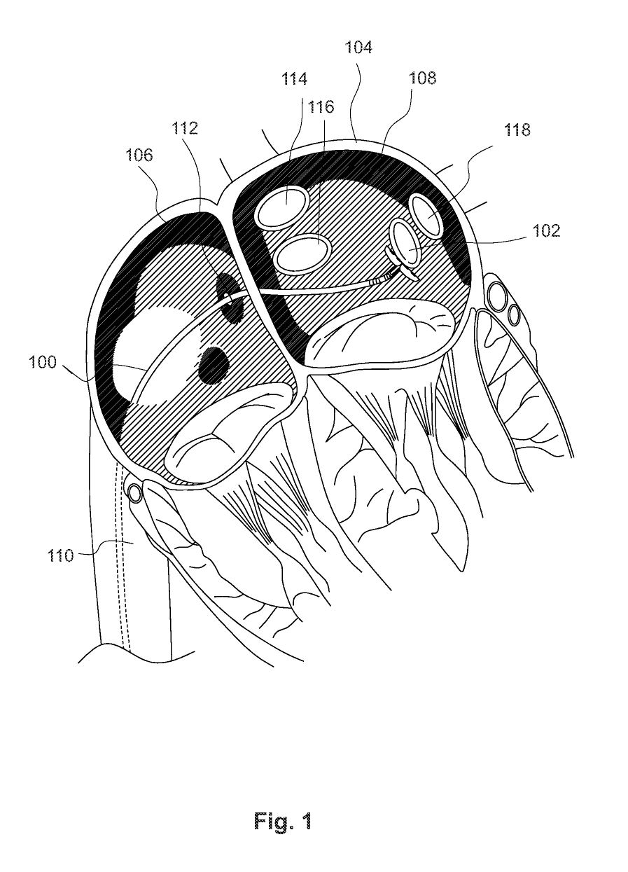

FIG. 1 is a schematic diagram illustrating a portion of a catheter device 100 positioned proximate a pulmonary vein 102 of a heart 104. As is known, the heart 104 includes a right atrium (RA) 106, a left atrium (LA) 108, the pulmonary vein 102, an inferior vena cava 110 and an intra-atrial septum 112. In various embodiments, the catheter device 100 is configured for use in ablating heart tissues to treat cardiac arrhythmias. In one embodiment, the catheter device 100 is configured for treating atrial fibrillation by ablating tissue surrounding the ostia of pulmonary veins 102 of the heart 104 and ensuring an electrical isolation of the pulmonary vein 102.

In the embodiment shown in FIG. 1, to position the catheter device 100 at a target tissue location in the pulmonary vein 102, the catheter device 100 can be inserted through a transseptal puncture. The transseptal puncture permits a direct route from the RA 106 to the LA 108 via an intra-atrial septum 112 (by puncturing the septum 112 proximate or at the fossa ovalis). Once, the catheter device 100 reaches the target tissue location in the pulmonary vein 102 in the LA 108 of the heart 104, the catheter device 100 is directed towards the pulmonary vein 102. The catheter device 100 can then be used to deliver radiofrequency (RF) energy to ablate the target tissues thereby isolating the pulmonary vein 102 from the rest of the heart 104 and preventing any pulses from the vein from getting into the heart 104. As shown, the LA 108 of the heart 104 includes three additional pulmonary veins 114, 116, and 118 that can be ablated as per the requirements in a manner similar to the ablation of the pulmonary veins 102.

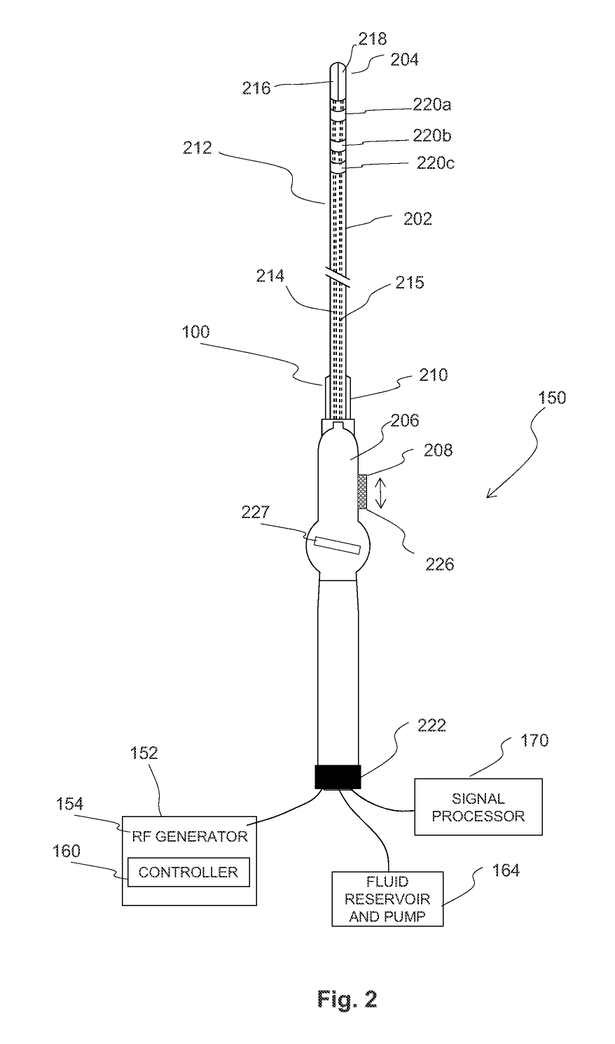

FIG. 2 is a schematic elevation view of an ablation system 150 including the catheter device 100, in an embodiment of the present invention. As shown in FIG. 2, in addition to the catheter device 100, the system 150 includes additional hardware and equipment including, in the particular embodiment shown, an ablation control system 152 including a radiofrequency generator 154 coupled to a controller 160, a fluid delivery system 164 including, among other things, a fluid reservoir and pump, and a signal processor 170. In various embodiments, for example, a system incorporating a non-irrigated catheter device 100, the fluid delivery system 164 can be omitted. In various embodiments, the ablation control system 152 is configured to provide a controlled amount of RF energy to the catheter device 100 as needed for the particular ablation procedure being performed. The RF controller 160 controls the timing and the level of the RF energy delivered through the catheter device 100. In the various embodiments, the signal processor 170 is configured, at least in part, to receive and process cardiac signals obtained by the catheter device 100 for interpretation and use by the clinician during the ablation procedure. The signal processor 170 can be configured to detect, process, and record electrical signals within the heart 104. Based on the detected electrical signals, the signal processor 170 outputs electrocardiograms (ECGs) to a display (not shown), which can be analyzed by the operator to determine the existence and/or location of arrhythmia substrates within the heart 104 and/or determine the location of the catheter device 100 within the heart 104. In an embodiment, the signal processor 170 generates and outputs an isochronal map of the detected electrical activity to the display for analysis by the operator. Although the ablation control system 152, the fluid delivery system 164, and the signal processor 170 are shown as discrete components, they can alternatively be incorporated into a single integrated device.

It is emphasized that the particular configuration and presence of the ablation control system 152, the fluid delivery system 164 and the signal processor 170 are not critical to the various embodiments. Thus, when present, any such systems and hardware, whether currently known or later developed, can be utilized within the system 150.

As further shown in FIG. 2, the catheter device 100 includes an elongate body 202, a tip section 204, a handle 206, and a control mechanism 208. Additionally, the elongate body 202 includes a proximal end 210, an opposite distal end 212, and a pair of inner fluid lumens 214, 215. As shown, the inner fluid lumens 214, 215 extend from the proximal end 210 through the distal end 212 of the elongate body 202. In various embodiments, the elongate body 202 is generally tubular and houses additional components including, without limitation, electrical conductors and, as will be discussed in greater detail below, components for manipulating the catheter device 100, including the tip section 204, during the ablation procedures.

In the illustrated embodiment, the tip section 204 of the catheter device 100 includes a first jaw member 216 and a second jaw member 218. In addition, in the particular embodiment shown, the distal portion of the catheter device 100 includes a plurality of ring electrodes 220a, 220b, 220c along the distal end 212 of the body 202 that can be used to gather electrocardiogram data before, during and after the particular ablation procedure. Additionally, as will be explained in further detail, a portion of the control mechanism 208 extends to and is operatively coupled to the jaw members 216, 218 to facilitate manipulation thereof during the procedure.

As further shown, the handle 206 is coupled to the proximal end 210 of the elongate body 202, and includes a connection port 222, and a portion of the control mechanism 208 (in the illustrated embodiment, a control element 226). The connection port 222 is operable to allow external devices and hardware, e.g., the ablation control system 152, the fluid delivery system 164 and/or the signal processor 170, to be operably coupled to the catheter device 100. In addition, the handle 206 further includes a plurality of conduits, conductors, and wires (not shown) to facilitate control of the catheter device 100. In the illustrated embodiment, the handle 206 also includes a control knob 227 operably to be manipulated by the clinician to deflect the distal end 212 of the elongate body 202. As such, the control knob 227 is mechanically and operably coupled to additional components (e.g., one or more control wires) extending along the elongate body 202. It is emphasized, however, that the particular technique for controlling deflection and steerability of the catheter device 100 is not critical to the various embodiments of the present invention. In addition, in various embodiments, the catheter device 100 is a fixed-shape catheter (i.e., is not steerable) and thus the control knob 227 and associated components can be omitted in such embodiments.

The tip section 204 is formed from an electrically conductive material. For example, some embodiments may use a platinum-iridium alloy. Some embodiments may use an alloy with approximately 90% platinum and 10% iridium.

The elongate body 202 can, in various embodiments, range from about 1.67 millimeters to about 3 millimeters in diameter, and between about 800 millimeters and 1500 millimeters in length. The foregoing dimensions, however, are exemplary only, and can vary depending on the particular clinical needs for the catheter device 100.

In various embodiments, the elongate body 202 can have a circular cross-sectional geometry. However, other cross-sectional shapes, such as elliptical, rectangular, triangular, and various other shapes, can be provided. In some embodiments, the elongate body 202 can be preformed of an inert, resilient polymeric material that retains its shape and does not soften significantly at body temperature; for example, polyether block amides, polyurethane, polyester, and the like. The elongate body 202 can be flexible so that it is capable of winding through a tortuous path that leads to a target site. In some embodiments, the elongate body 202 can be semi-rigid, i.e., by being made of a stiff material, or by being reinforced with a coating, braid, coil, or similar structure, to control the flexibility of the elongate body 202.

The handle 206 is used to be comfortably held by an operator during a treatment procedure involving ablation. The handle 206 is composed of a durable and generally rigid material, such as medical grade plastic, and ergonomically molded to allow the practitioner to easily manipulate the catheter device 100.

In various embodiments, the jaw members 216, 218 are configured to be operable as RF ablation electrodes to deliver RF energy to target cardiac tissue in an RF ablation procedure. As shown, the tip section 204 is positioned at the distal end 212 of the elongate body 202 and the handle 206 is positioned at the proximal end 210 of the elongate body 202. As will be explained in further detail herein, a portion of the control mechanism 208 is placed at the tip section 204 and a portion of the control mechanism 208 is placed at the handle 206 of the catheter device 100. The control mechanism 208 is configured to manipulate the tip section 204 between a closed and an open configuration. In the closed configuration, the jaw members 216 and 218 are generally parallel to the elongate body 202. In an embodiment, in the open configuration, the jaw members 216 and 218 can be configured to lie substantially perpendicular to the elongate body 202. In the various embodiments, the tip section 204 can be operable as an RF ablation electrode in both the open and closed configurations.

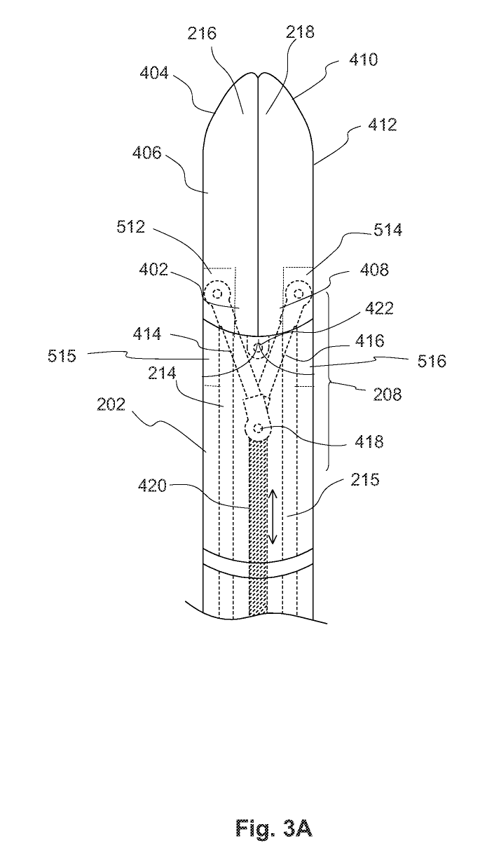

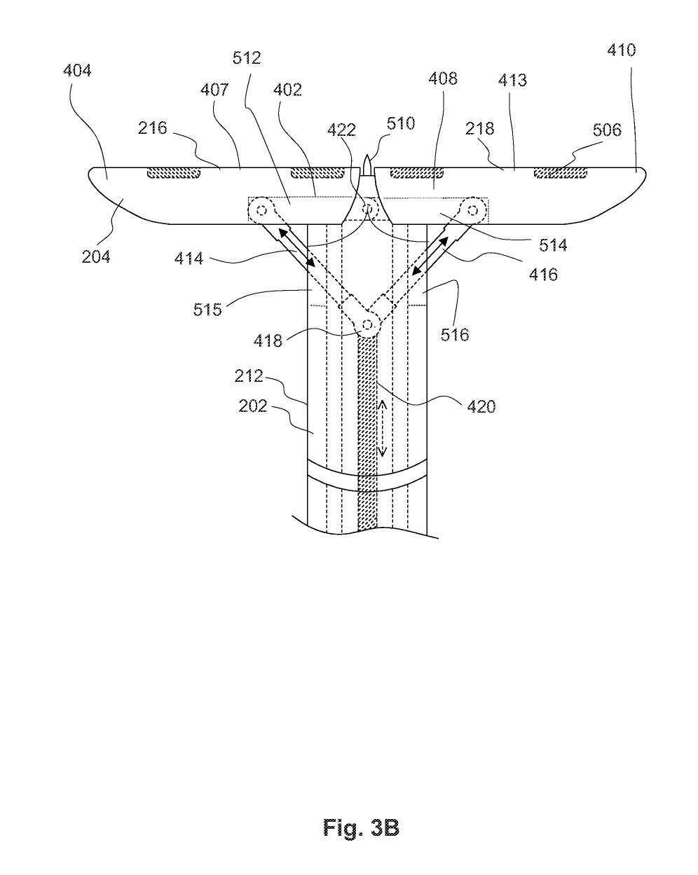

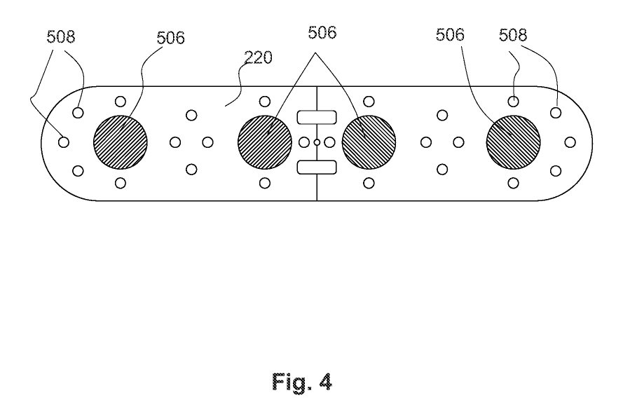

FIGS. 3A and 3B are schematic partial elevation views of the distal portion of the catheter device 100 showing the tip section 204 in the closed and open configurations, respectively. In addition, FIG. 4 is a schematic end view of the tip section 204 in its open configuration. As shown in FIG. 3B, the first jaw member 216 includes a first proximal portion 402, a first distal portion 404, a first outer surface 406, and a first inner surface 407. As further shown, the second jaw member 218 includes a second proximal portion 408, a second distal portion 410, a second outer surface 412, and a second inner surface 413.

In the illustrated embodiment, the control mechanism 208 includes a first control member 414 and a second control member 416. In some embodiments, the control mechanism 208 can include additional elements to facilitate manipulation and actuation of the jaw members 216, 218, such as one or more pivot pins 418, a control wire 420 and the like. As shown, the first and second proximal portions 402, 408 of the jaw members 216, 218 are pivotally connected to one another via a hinge element 422. In addition, the first and second control members 414, 416 extend partially within the elongate body 202 and are also connected to the first and second proximal portions 402, 408 of the first and second jaw members 216 and 218, and also to the control element 226 (e.g., via the control wire 420 and the pivot pin 418).

The control wire 420 can be made of any polymeric or metallic material having sufficient flexibility and mechanical strength to transfer forces between the control element 226 and the tip section 204. The diameter and the constituent material of the control wire 420 are selected to ensure that the control wire 420 has sufficient axial stiffness to support the axial load necessary to open the jaws. Exemplary materials include, without limitation, nickel titanium (nitinol) alloys, stainless steels, and the like. In one embodiment, the control wire 420 can be made of nitinol and can have a diameter of about 0.018-0.019 inch. The control wire 420 operably connects the jaw members 216 and 218 with the control element 226. In various embodiments, the control element 226 can be a knob, or a push button or any other similar element.

As shown, in the open configuration of the tip section 204, the inner surfaces 407, 413 lie substantially in the same plane and the jaw members 216, 218 are oriented generally orthogonal to the elongate body 202. In the various embodiments, the inner surfaces 407, 413 are operable as RF ablation electrodes for forming linear ablation lesions in or on the target cardiac tissue. In an embodiment, the inner surfaces 407, 413 of the first and second jaw members 216 and 218 are generally flat.

As can be further seen in FIGS. 3B and 4, in the illustrated embodiment, the tip section 204 includes a plurality of microelectrodes 506, a plurality of irrigation ports 508, and a locking element 510. As shown, the micro-electrodes 506 and the irrigation ports 508 are disposed along the inner surfaces 407, 413 of the jaw members 216, 218, respectively, with at least one microelectrode 506 and at least one irrigation port 508 in each of the jaw members 216, 218. As can be seen in FIGS. 3B and 4, when the tip section is in its open configuration, the microelectrodes 506 and the irrigation ports 508 will be exposed to the environment.

In addition, the jaw member 216 includes a first slot 512 and the jaw member 218 includes a second slot 514. As further shown, the distal end 212 of the body includes slots 515, 516 aligned, respectively, with the first slot 512 and the second slot 514.

In an embodiment, the slots 512, 515 are configured to receive the first control member 414, and the slots 514, 516 are configured to receive the second control member 416. This arrangement allows the control members 414, 416 to translate and rotate relative to the elongate body 202 to facilitate transitioning the tip section 204 between its closed and open configurations.

In use, the operator can manipulate the catheter device 100 so as to direct and position the catheter tip section 204 at the target tissue location (see, e.g., FIG. 1). Upon reaching the target tissue location (i.e. a location proximate the pulmonary vein to be isolated), the catheter device 100 can be used in a generally conventional manner to map and/or ablate cardiac tissue. For example, in the illustrated embodiment, the ring electrode 220a and the tip section 204, in the closed configuration, can be used as a pair of mapping electrodes. Similarly, tissue can also be mapped between the electrode pair 220b, 220c. Additionally, the tip section 204 can be used as either a point ablation source (i.e., by positioning the tip section 204 to lie generally perpendicular to the tissue to be ablated), or as a linear ablation electrode (i.e., by manipulating the catheter device 100 so that the outer surface of the tip section 204 lies along the tissue to be ablated.

Subsequently, the clinician can manipulate the control element 226 to cause the control members 414 and 416 to move relative to the elongate body 202 so as to cause the tip section 204 to transition from its closed configuration to its open configuration. In the closed configuration, the inner surfaces 407, 413 are at least partially in contact with one another.

In order to transition the tip section 204 from the closed configuration to the open configuration, a force is applied on the control element 226. Upon application of the force on the control element 226, the pulling of the control wire 420 causes deflection of the first and second control members 414 and 416. Upon application of the force, the hinge element 422 facilitates transition of the tip section 204 from the closed configuration to the open configuration. The hinge element 422 facilitates splitting of the tip section 204 by allowing the distal portions 404 and 410 of the first jaw member 216 and the second jaw member 218 to rotate and deflect away from one another. In various other embodiments, the hinge element 422 also includes linkages to allow the first and second proximal portions 402, 408 to deflect laterally away from one another as the tip section 204 opens, to further facilitate transition to the open configuration.

In some embodiments, the tip section 204 can be returned to its closed configuration by further manipulation of the control mechanism 208. In various embodiments, the jaw member 216, 218 are biased (e.g., by a spring or similar element, not shown, in the control mechanism 208) toward the closed configuration. In such embodiments, upon removal of the force, the jaw members 216 and 218 can automatically transition back to the closed configuration.

In various embodiments, the locking element 510 is operable to retain the tip section 204 in the desired position relative to the target cardiac tissue. In the illustrated embodiment, the locking element 510 can be a pin or similar structure configured to engage the tissue to inhibit unintended movement of the tip section 204 during formation of the ablation lesion. The first and the second jaw members 216 and 218 are configured to revolve on the locking element 510, which is exposed when the tip section 204 assumes its open configuration.

In the open configuration, the inner surfaces 407, 413 of the first jaw member 216 and the second jaw member 218 are exposed to the target tissue location, and can be operable as RF ablation electrodes to form a generally linear ablation lesion on the targeted cardiac tissue. In addition, the microelectrodes 506 are exposed to the target tissues that allow the operator to map the electrical signals within the heart 104. The electrical signals are recorded via the microelectrodes 506 of the catheter device 100. The electrical signals within the heart 104 can be detected, processed, and recorded by the signal processor 170 that is coupled to the microelectrodes 506. Based on the electrical signals sensed by the microelectrodes 506, the operator can identify the specific target tissue sites within the heart 104, and ensure that the arrhythmia causing substrates have been electrically isolated by the ablative treatment. After mapping the electrical signals and upon recognition of the tissues responsive for causing arrhythmia, the operator can then ablate the target tissue, with the inner surfaces 407, 413 of the jaw members 216, 218 in contact with the tissue and operable as the RF ablation electrodes using the energy generated from the ablation control system 152. In various embodiments, the irrigation ports 508 are fluidly coupled to the fluid lumens 214, 215. In order to maintain the temperature of the tip section 204 and surrounding tissues during ablation, a cooling fluid, such as a saline fluid, is delivered from the fluid delivery system 164 through the fluid lumens 214, 215 to the catheter tip section 204, where the fluid exits through irrigation ports 508 to cool the tip section 204 and surrounding tissue. In an embodiment, the inner fluid lumens 214, 215 can be formed of a flexible material so as to get articulated during transition between the open configuration and the closed configuration. In the illustrated embodiment, the fluid lumen 214 is fluidly coupled to and supplies cooling fluid to the irrigation ports 508 in the jaw member 216, while the fluid lumen 215 is fluidly coupled to and supplies cooling fluid to the irrigation ports 508 in the jaw member 218. This arrangement is illustrative only, however, and so other fluid lumen and irrigation port configurations can be utilized within the scope of the various embodiments. In various embodiments, the microelectrodes 506 and/or the fluid/coolant delivery capabilities are omitted from the system.

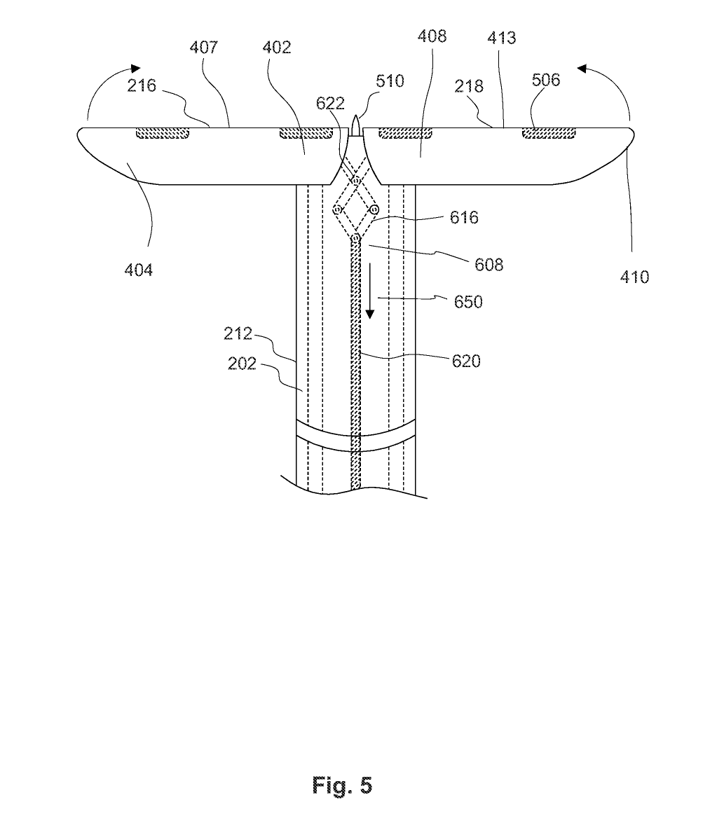

The mechanism shown in the figures is exemplary and modifications may be made for employing a different control mechanism 208 that is capable of causing the jaw members to transition between the open and closed configurations. For example, FIG. 5 is a schematic elevation view of the distal end portion of an alternative embodiment of the catheter device 100 with the tip section 204 in its open configuration. In the particular embodiment shown in FIG. 5, the catheter device 100 includes an alternative control mechanism 608 including a control linkage 616 and a control wire 620. As further shown, the control linkage includes an arrangement of linking members pivotally connected to one another, including at a fixed pivot hinge 622. As further shown, the linkages are connected to the jaw members 216, 218. In the illustrated embodiment, the control wire 620 is shown in its distal-most position such that the jaw members 216, 218 lie relatively orthogonal to the longitudinal axis of the catheter body 202. As indicated by the arrow 650 in FIG. 5, proximal motion of the control wire 620 relative to the body 202 (accomplished by, for example, manipulation of the control element 226 shown in FIG. 2) will cause the distal end portions 404, 410 of the jaw members 216, 218 to rotate toward one another about the fixed pivot hinge 622, thus transitioning the tip section 204 to its closed configuration. Of course, still other actuation mechanisms may be employed to facilitate transitioning the tip section 204 between its open and closed configurations.

Clinical benefits of the catheter device 100, in some embodiments, can include, but are not limited to, controlling the temperature and reducing coagulum formation on the tip section 204 of the catheter, preventing impedance rise of tissue in contact with the catheter tip, and maximizing potential energy transfer to the tissue. Additionally, the localized intra cardiac electrical activity can be recorded in real time or near-real time right at the point of energy delivery. The open configuration of the tip section 204 allows the operator to perform the longer ablations by doubling the active length of the ablation electrode as compared to a conventional RF tip electrode. At the same time, the compact profile of the tip section 204 when in the closed configuration facilitates ease of delivery and deployment of the catheter device 100 to the area of interest within the heart 104.

I claim:

1. A catheter device for use in ablating heart tissues, the catheter device comprising:
an elongate body having a proximal end and an opposite distal end; and
a tip section positioned at the distal end of the elongate body, the tip section including a first jaw member and a second jaw member, each of the jaw members including a proximal portion, a distal portion, an outer surface, and an inner surface, the jaw members pivotally joined to one another at the proximal portions thereof, the tip section configured to transition between a closed configuration in which the inner surfaces are at least partially in contact with one another, and an open configuration in which the distal portions of the jaw members are deflected away from one another,
wherein the tip section is operable in both the open and closed configurations as an ablation electrode for selectively ablating the heart tissues.

2. The catheter device of claim 1, wherein the proximal portions of the first jaw member and the second jaw member are coupled together by a hinge element to allow splitting of the tip section by allowing the distal portions of the first jaw member and the second jaw member to deflect away from one another.

3. The catheter device of claim 1, wherein the catheter is an irrigated catheter configured to deliver irrigation fluid through the tip section during an ablation procedure.

4. The catheter device of claim 1, wherein the tip section is configured to map electrical activity of the heart.

5. The catheter device of claim 1, wherein the first jaw member and the second jaw member are substantially perpendicular with respect to the elongate body in the open configuration.

6. The catheter device of claim 5, wherein the first jaw member and the second jaw member configured to lie linearly in the open configuration.

7. The catheter device of claim 1, wherein the inner surfaces of the first and second jaw members are generally flat.

8. The catheter device of claim 1, further comprising a control mechanism coupled to the tip section for manipulating the tip section between its closed and open configurations.

9. The catheter device of claim 8, wherein the control mechanism includes first and second control members connected, respectively, to the first and second jaw members and slideably disposed within the elongate body.

10. The catheter device of claim 8, further comprising a handle coupled to the proximal end of the body, wherein the control mechanism includes a control element on the handle, and wherein the first and second control members extend at least partially within the elongate body from the tip section to the handle and are coupled to the control element, and wherein manipulation of the control element causes the control members to move relative to the elongate body so as to cause the tip section to transition between its open and closed configuration.

11. The catheter device of claim 10, wherein the control mechanism is configured such that proximal movement of the first and second control members relative to the elongate body causes the distal portions of the first and second jaw members to deflect away from one another thereby causing the tip section to assume its open configuration.

12. The catheter device of claim 8, wherein the control mechanism includes a locking element to releasably retain the tip section in its open configuration at the target location within the patient.

13. A catheter device for use in ablating heart tissues, the catheter device comprising:
an elongate body having a proximal end and an opposite distal end, and an inner fluid lumen extending from the proximal end through the distal end of the body; and
a tip section positioned at the distal end of the elongate body, the tip section including:
a first jaw member and a second jaw member, each of the jaw members including a proximal portion, a distal portion, an outer surface, and an inner surface, the jaw members pivotally joined to one another at the proximal portions thereof, the tip section configured to transition between a closed configuration in which the inner surfaces are at least partially in contact with one another, and an open configuration in which the distal portions of the jaw members are deflected away from one another, wherein the tip section is operable as an ablation electrode for selectively ablating the heart tissues;
a plurality of microelectrodes positioned on the inner surface of each of the jaw members for sensing electrical signals originating from the heart tissues; and
a plurality of irrigation ports provided on the inner surface of each of the jaw members and in fluid communication with the fluid lumen.

14. The catheter device of claim 13, wherein, the first jaw member and the second jaw member are substantially perpendicular with respect to the elongate body in the open configuration.

15. The catheter device of claim 14, wherein the first jaw member and the second jaw member configured to lie linearly in the open configuration.

16. The catheter device of claim 13, wherein the inner surfaces of the first and second jaw members are generally flat.

17. The catheter device of claim 13, further comprising a control mechanism coupled to the tip section for manipulating the tip section between its closed and open configurations, and further wherein the tip section is biased to its closed configuration, and wherein manipulation of the control mechanism causes the tip section to assume its open configuration.

18. A tissue ablation method comprising:
advancing a portion of a catheter device to a location proximate target tissue within a patient's heart, wherein the catheter device includes a tip section positioned at a distal end of an elongate body of the catheter device, the tip section including a first jaw member and a second jaw member, each of the jaw members including a proximal portion, a distal portion, an outer surface, and an inner surface, the jaw members pivotally joined to one another at the proximal portions thereof, wherein the advancing step is performed with the tip section in a closed configuration in which the inner surfaces are at least partially in contact with one another;
manipulating a control mechanism on the catheter device to cause the distal portions of the jaw members to deflect and rotate away from one another such that the tip section assumes an open configuration wherein the inner surfaces of the jaw members are exposed to and positioned proximate the target tissue;
positioning the inner surfaces of the jaw members in contact with the target tissue; and
causing RF ablation energy to be supplied to the jaw members to effectuate RF ablation of the target tissue in contact with the inner surfaces of the jaw members.

19. The method of claim 18, further comprising, after manipulating the control mechanism to cause the tip section to assume its open configuration, mapping cardiac electrical activity proximate the target tissue using microelectrodes positioned on the inner surfaces of the jaw members.

20. The method of claim 19, further comprising causing irrigation fluid to be supplied to the target tissue while supplying the RF ablation energy via irrigation ports disposed in the inner surfaces of the jaw members.
